(19) United States
(12) Patent Application Publication
(10) Pub. No.: US 2004/0092896 A1
(43) Pub. Date: May 13, 2004

(54) Antimicrobial sheets for environmental and human protection

(76) Inventor: Kaila R. Thompson, Naples, FL (US)

Correspondence Address: Merrill N. Johnson, 963 Trail Terrace Drive, Naples, FL, 34103, US

(21) Appl. No.: 10/293,546

(22) Filed: Nov. 13, 2002

(51) Int. Cl. A61F 13/00

(52) U.S. Cl. 604/304

(57) ABSTRACT

Antimicrobial sheets for environmental and human protection. Preferably the sheets are formed into rings about three inches in diameter, three quarters of an inch wide and one eighth of an inch thick to be continuously worn on a person's wrist.

### FIELD OF THE INVENTION

The invention lies in the field of the protection of human life.

### SUMMARY OF THE INVENTION

Applicant's antimicrobial sheets are preferably in the form of a three quarters of an inch wide and one eighth of an inch thick tape having on one side or on both sides an antimicrobial compound. The tapes are are formed into a ring having a diameter of about three inches. The ring is designed to be continuously worn on the wrist of both healthy people and people suffering from some disease or other abnormality. The antimicrobial material on the ring will cure those people suffering from some abnormality and it will protect healthy people from becoming ill.

### DESCRIPTION OF THE DRAWING

FIG. 1 is a ring of antimicrobial tape to be continuously worn on the wrist.

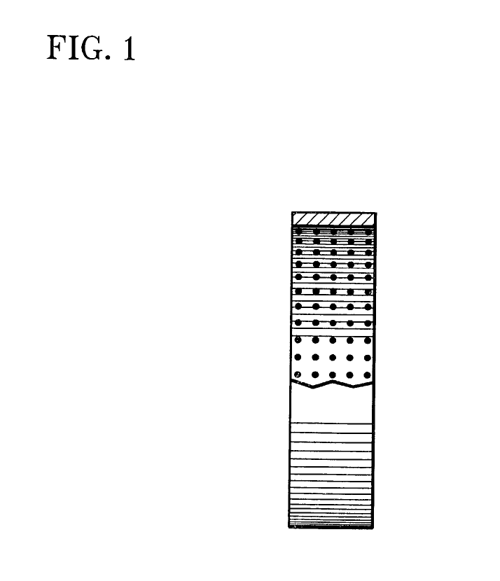

### DETAILED DESCRIPTION OF THE PREFERRED FORM OF THE INVENTION

Applicant's ring of tape may contain one or more of several antimicrobial compounds, some of which are presently used in commercially known plastics and soaps. Two of the compounds known to be used in plastics and soaps are triclosan (2,4,4-trichloro-2hydroxy-diphenylether) and n-alkyl dimethylbenzyl ammonium chloride dimethyl benzyl ammonium chloride.

U.S. Pat. No. 5,783,209 which issued Jul. 21, 1998, was assigned prior to its issue to Minnesota Mining and Manufacturing Company (MMM). U.S. Pat. No. '209 teaches medical pressure-sensitive dressings which may or may not contain a number of antimicrobial compounds. It is not known if MMM markets medical dressings containing one or more antimicrobial compounds. However, based on the publication of U.S. Pat. No. 5,783,209 in 1998, its teaching is public knowledge.

While I have disclosed in this specification and shown in its drawing a detailed explanation of my invention, the true scope and extent of my invention is set forth only in the following claims.

I claim:

1. Antimicrobial sheets for environmental and human protection.

2. An antimicrobial sheet as set forth in claim 1 in which the sheet is formed into a three quarters of an inch wide and one eighth of an inch thick tape.

3. An antimicrobial tape as set forth in claim 2 which is formed into a ring having a diameter of three inches designed to be continuously worn on a person's wrist.

4. A tape covered by one or more of claims 1, 2 and 3 which is not limited to commercial and residential uses and is large enough to cover all types of surfaces that may contain harmful bacteria.
